(19) United States
(12) Patent Application Publication
(10) Pub. No.: US 2016/0194698 A1
(43) Pub. Date: Jul. 7, 2016

(54) SYSTEMS, APPARATUSES AND METHODS FOR READING POLYMER SEQUENCE

(71) Applicant: ARIZONA BOARD OF REGENTS on behalf of ARIZONA STATE UNIVERSITY, Scottsdale, AZ (US)

(72) Inventor: Stuart LINDSAY, Phoenix, AZ (US)

(21) Appl. No.: 14/971,393

(22) Filed: Dec. 16, 2015

Related U.S. Application Data
(60) Provisional application No. 62/092,792, filed on Dec. 16, 2014.

(51) Int. Cl. C12Q 1/68; G01N 27/447

(52) U.S. Cl. CPC C12Q 1/6869; G01N 27/44791

(57) ABSTRACT

A device for sequencing single polymer molecules. The device includes a molecular clamp bound to a first electrode so as to hold the polymer in place with respect to the first electrode, which can be in close proximity to a second electrode so that a signal characteristic of each molecular residue on the polymer is generated as the polymer passes the gap between the electrodes. A third electrode may be biased with respect to the first two electrodes so as to exert a force that pulls the molecule through the molecular clamp and past the junction between the first two electrodes.

### CROSS-REFERENCE TO RELATED APPLICATIONS

This applications claims priority to U.S. provisional application No. 62/092,792 titled “SYSTEMS, APPARATUSES AND METHODS FOR READING POLYMER SEQUENCE”, filed Dec. 16, 2015, the entire disclosure of which is incorporated herein by reference.

### SUMMARY

Embodiments of a device for sequencing single polymer molecules are disclosed herein. In some embodiments, the device comprises a molecular clamp bound to a first electrode so as to hold the polymer in place with respect to the first electrode, which can be in close proximity to a second electrode so that a signal characteristic of each molecular residue on the polymer is generated as the polymer passes the gap between the electrodes. In some embodiments, a third electrode may be biased with respect to the first two electrodes so as to exert a force that pulls the molecule through the molecular clamp and past the junction between the first two electrodes.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows an example embodiment of a manufacturing process for constructing a sequencing device according to some of the embodiments disclosed herein.

FIG. 2 shows an example schematic of a device disclosed herein comprising molecular, solid state and electronic elements.

FIG. 3 shows an example illustration of the functionalization of device electrodes according to some embodiments.

FIG. 4 shows an example embodiment of sample preparation for a DNA-helicase complex.

FIG. 5 shows an example embodiment of molecular components for reading single stranded DNA using a helicase to apply a braking force and an electric field to advance the polymer passing the electrodes.

FIG. 6 shows an example embodiment of molecular components for reading double stranded DNA using a helicase to apply a braking force and an electric field to advance the polymer passing the electrodes.

FIG. 7 shows an example embodiment of sample preparation for a protein-ClpX complex.

FIG. 8 shows an example embodiment of molecular components for reading protein sequence using an unfoldase motor and a protein modified at its N terminus with a series of charged residues and a binding recognition sequence for the unfoldase.

FIGS. 9A-B show example potential distributions near tunneling electrodes due to the placement of a collector electrode.

### BACKGROUND OF THE INVENTION

Embodiments of a method for manufacturing a tunnel junction such that individual molecular species give distinct electronic signals when in contact with recognition molecules bound to the electrodes that comprise the tunnel junction have previously been discussed. For example, U.S. Patent Publication No. 2014/0113386, titled “Systems and Methods for Molecule Sensing and Method of Manufacturing Thereof,” filed on Oct. 10, 2013, and “Fixed Gap Tunnel Junction for Reading DNA Nucleotides” by P. Pang et al., ACS Nano, published Nov. 7, 2014 (online), the entire contents of which are incorporated by reference herein, disclose said embodiments. The disclosed embodiments further comprise methods of cutting a nanopore through the layers that comprise the junction, so that each molecular unit (e.g., DNA base, protein residue or sugar molecule in an oligosaccharide, etc.) can be read as the unit passes the electrodes embedded in the nanopore.

However, in some implementations, it may be challenging to cut nanopores through closely spaced electrodes without damaging them. For example, one method of cutting nanopores, reactive ion etching (ME), may produce cuts through a stack of electrodes with quite a high yield of working devices, yet the nanopores may be limited to diameters of 20 nm or above due to masks required for the RIE (e.g., see “Fabrication of sub-20 nm nanopore arrays in membranes with embedded metal electrodes at wafer scales” by Bai et al., Nanoscale, published Mar. 7, 2014 (online)). As another example, He-ion focused ion beam (FIB) can cut holes as small as 5 nm in diameter, but the yield may not be high. In addition, it may be challenging to scale up production of devices made with a single FIB as each hole may be drilled individually.

In some implementations, controlling the translocation of polymers through a nanopore may prove to be another challenge. WIPO Patent Application No. WO/2014/138253, titled “Translocating a Polymer through a Nanopore”, filed Mar. 5, 2014, the entire contents of which is incorporated by reference herein, discloses a universal process for ligating a charged tail to a protein of arbitrary charge, so that proteins can be pulled into a nanopore device by electrophoresis. In some embodiments, DNA, which can be naturally charged, may not require such modifications. In some instances, even in a small nanopore where the polymer may be held close to the recognition molecules, the interactions can be stochastic, leading to a very wide distribution of times for which a target molecule may be captured. In addition, as described in Krishnakumaer et al., titled “Slowing DNA Translocation through a Nanopore Using a Functionalized Electrode”, ACS Nano, published Oct. 28, 2013, the entire contents of which are incorporated by reference herein, many of the molecules can pass through the pore without binding to the recognition molecules on an electrode.

Further, in the case of DNA, because contact with a single base may generate a recognizable tunnel current signature, the schemes for readout of sequence by means of electron tunnel current may take place for single stranded DNA. In some instances, single stranded DNA may be much harder to handle and prepare than double stranded DNA, which may be particularly true for the kilobase or more of DNA that would constitute a long sequence read.

Accordingly, a device that altogether dispenses with the need for a small nanopore may be welcome. Furthermore, it may be desirable to have a device that can control the flow of the polymer by means of forces applied to the polymer across the part of the device where the sequence is read. In addition, in the case of DNA, it is desirable to have a device that operates on double-stranded DNA.

### DETAILED DESCRIPTION OF SOME OF THE EMBODIMENTS

At least some of the embodiments solves at least some of the problems discussed above and realize certain advantages. In some embodiments, the device uses a molecular clamp bound to a first electrode, so as to hold the polymer in place with respect to the first electrode, in close proximity to a second electrode so that a signal characteristic of each molecular residue on the polymer is generated as the polymer passes the gap between the electrodes. A third electrode is biased with respect to the first two electrodes so as to exert a force that pulls the molecule through the molecular clamp and past the junction between the first two electrodes. Such embodiments lend improvements to the device, systems and methodology disclosed in “Hybrid pore formation by directed insertion of alpha hemolysin into solid-state nanopores” by A. R. Hall, et al., Nature Nanotechnology, Nov. 28, 2010. To that end, embodiments disclosed herein can form still other embodiments when taken in combination with one or another of the disclosed devices, systems and methods disclosed in the Hall et al. publication (herein incorporated by reference). For example, some embodiments of the present disclosure lack the requirement of a constraining nanopore as required by Hall et al.

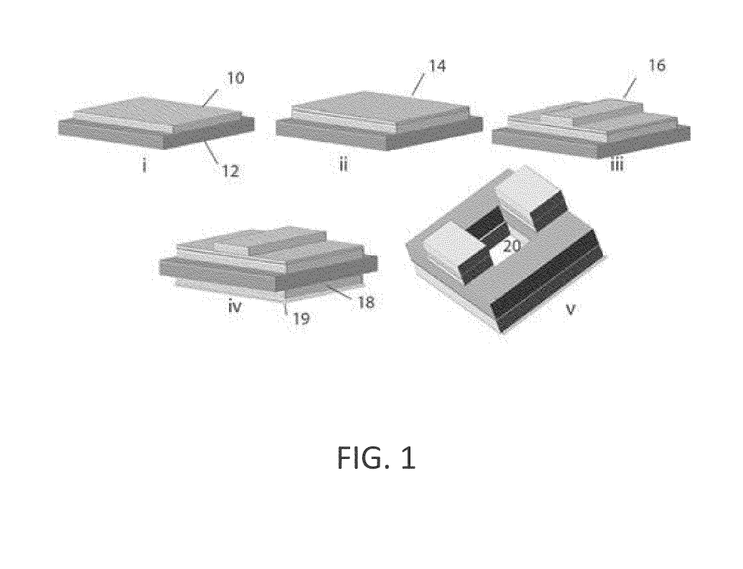

With reference to FIG. 1, in some embodiments, a process for fabricating solid state components of a sequencing device is disclosed. FIG. 1 shows the steps involved in making one device, but it will be recognized to those skilled in the art that many such devices can be made on a wafer, each individually addressed by methods well known in the manufacture of computer memory devices. In some instances, e.g., at step (i), a thin layer (e.g., ranging 1 to 100 nm in thickness) of a noble metal 10 may be deposited on a thin membrane (e.g., ranging 1 to 100 nm in thickness) made of a dielectric material 12. Examples of the noble metal comprise palladium (Pd), platinum (Pt), gold (Au), and/or the like. Examples of a dielectric material comprise silicon nitride and/or oxides of silicon. At step (ii), another thin layer of dielectric insulator 14 (e.g., ranging 0.5 to 5 nm in thickness—more particularly 2 nm, for example) may be deposited. An example of a dielectric insulator may be aluminum oxide deposited by atomic layer deposition (ALD). In some instances, the thin layer may be other metal and semiconductor oxides, such as but not limited to, oxides of silicon or hafnium.

At step (iii), a top electrode 16 is deposited on top of the dielectric layer 14, using again a thin layer (e.g., ranging 1 to 100 nm in thickness) of noble metal. At step (iv), an additional electrode 18 ranging in thickness from 1 nm to several microns may be deposited on the underside of the membrane 12. As an example, such an electrode may be made from one of the noble metals listed above. Further, the electrode 18 may be covered with a layer of passivating material 19 on its underside, examples of such material comprising any insulator such as, but not limited to poly (methyl methacrylate) (PMMA), silicon oxide, etc. At step (v), a cut 20 may be made through the whole assembly. Examples of techniques that may be utilized to make such cuts are RIE, FIB cutting, etc. In some instances, the RIE may comprise of cycles that selectively remove the top metal 16, the dielectric layer 14, the bottom metal 10, the substrate material 12, the bottom electrode metal 18 and/or the bottom passivation layer 19. In some instances, the removal of the materials may be in order, for example, starting with a cycle that removes top metal 16, followed by the dielectric layer 14, followed by the bottom metal 10, subsequently followed by the substrate material 12, followed by the bottom electrode metal 18 and finally the bottom passivation layer 19 where it is exposed through the opening 20. The lateral size of the opening 20 may be defined by a mask formed lithographically on top of the device, where the mask material can be an about micron thick layer of PMMA and/or silicon oxide. This masking layer may be left in place to minimize the area of the top electrode 16 that contacts electrolyte solution. In some instances, the lateral extent of this orifice may not be constrained by any requirement, because its role may not be to confine the polymer. In some embodiments, its diameter may be limited to a dimension across the opening of about 20 nm.

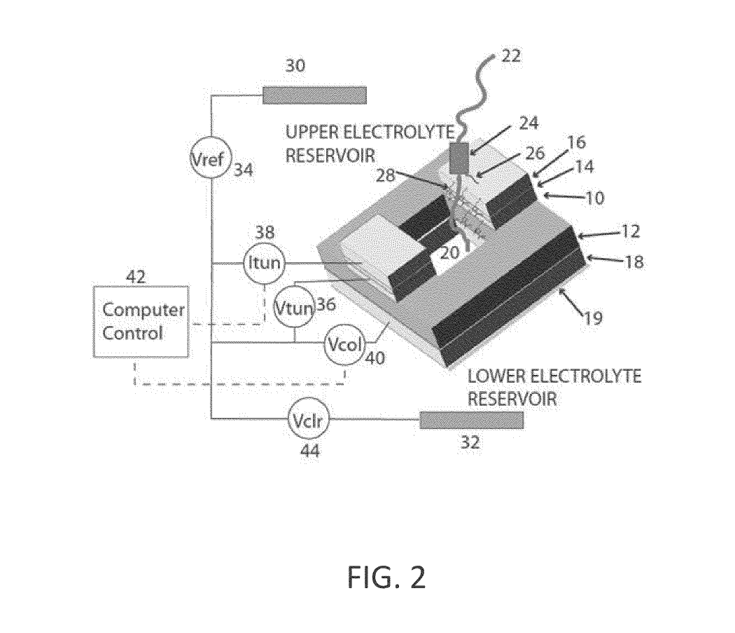

With reference to FIG. 2, in some embodiments, confinement of a polymer to be sequenced may be achieved, for example, by using a molecular ring that specifically binds the target polymer and that can be attached near the edge of the tunnel junction. The top two electrodes (16, 10) are electrically connected across the device, but shown in FIG. 2 as separate so that the orifice 20 can be seen clearly. The device serves as partition between two reservoirs of electrolyte, one in contact with a top reference electrode 30 and the second in contact with a lower reference electrode 32 with the only current path between the two reservoirs being through the opening in the device 20. In some instances, the polymer 22 to be sequenced may be bound with a molecular ring 24 that may be, in turn, bound to the top electrode 16 by means of a tether 26. An example of such a tether may be a bifunctional linker such as any short flexible polymer containing reactive groups at both ends (see, for example, the products sold by Creativepegworks www.creativepegworks.com). For example, a thiol at one end may bind to the metal electrode 16, while the other end can be a vinyl sulfone or maleimide deigned to bind to cysteines (sulfurs) on the ring molecule. As another example, lysine residues can be bound using bromo-acetyltion of amines as described in WIPO Patent Application No. WO 2014/190299, titled “Improved Chemistry For Translocation Of A Polymer Through A Nanopore”, filed May 23, 2014, the entire contents of which is incorporated by reference herein. In some instances, N-hydroxysuccinimide (NHS) esters and/or an antibody, raised to peptide motifs on the ring molecule, may also be used.

In such instances, the polymer may be held close to the edge of the reading device without necessarily having a built-in nanopore. The electrodes may be functionalized with appropriate recognition molecules 28 as described by the afore-mentioned publication by Pang et al. An example of the molecular ring 24 can be a molecular motor that unwinds DNA, such as but not limited to a helicase, polymerase, topoisomerase, etc. In the case of a protein, an unfoldase such as ClpX, (discussed Nivala et al., titled “Unfoldase-mediated protein translocation though an alpha-hemolysin pore”, Nature Biotechnology, published Feb. 3, 2013), one of the proteasomes (e.g., 19S available from UBP Bio (http://www.ubpbio.com/index.php/) may be used as molecular ring 24. The ring can also be passive, such as the polymerase clamp assembly. Organic molecules such as cyclodextrins, discussed by Ashcroft et al., in “An AFM/Rotaxane Molecular Reading Head for Sequence-Dependent DNA Structure”, Small, published September 2008, could also function in this capacity.

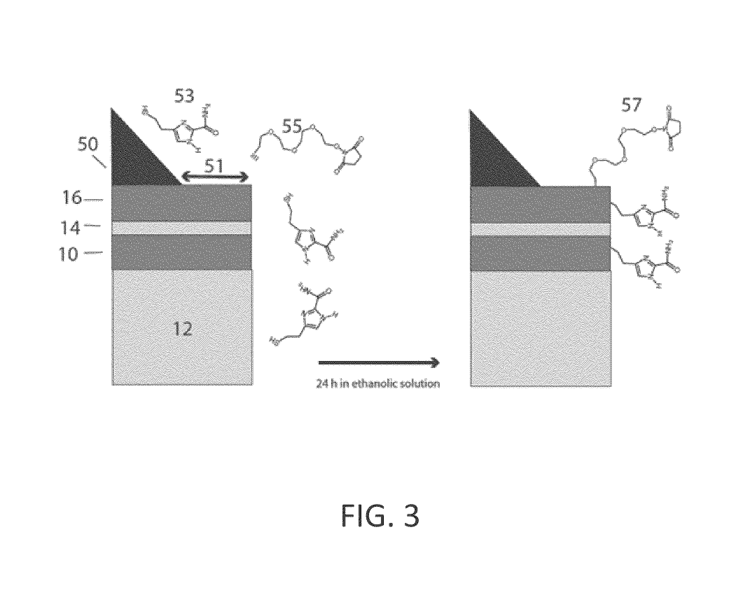

With reference to FIG. 3, in some embodiments, an example preparation of a device for chemical modification is shown. In some instances, as described above, a corner of the top electrode 16, which may be a significant part of the electrode, may be left uncovered by the passivation layer 50 used as a mask for the RIE because of the effect of the RIE etching on the mask materials, resulting in an uncovered area 51. The device may be incubated with an ethanolic solution of the recognition molecules 53 containing a small fraction of bifunctional linkers 55, an example of which is the thio-PEG-NHS molecule (see, for example, www.nanocs.net) shown in FIG. 3. After exposure to this solution for about 24 h, most of the electrode area will be covered in recognition molecules with a small fraction also functionalized with the bifunctional linker. Because the top electrode offers the most exposed area, it may be likely that the linker will occupy a site on the top electrode, e.g., 57.

In some instances, the functionalization of the top electrode 16 with ring molecules 24 that capture the target polymer may be largely random. However, many binding sites may be available across the top of an electrode that extends for many tens of nm (in one embodiment, the lateral extent of the top electrode is 50 nm), and the probability that one of the tethered rings may lie in close enough proximity so that the emerging polymer may interact with the tunnel junction formed by the electrodes 10 and 16, the dielectric layer 14 and the recognition molecules 28 can be substantial. In such instances, the polymer to be sequenced may be held close to the top electrode of the reading tunnel junction.

In some implementations, for such devices to operate stably, the electrodes may all be at potentials that lie outside regions where oxidation or reduction reactions occur in the solutions of molecules used. This may be achieved by connecting one electrode to a reference electrode in contact with the solution, as described in U.S. Patent Application Ser. No. 61/944,322, titled “Methods and Apparatuses for Stabilizing Nano-Electronic Devices in Contact with Solutions”, filed Feb. 25, 2014, the entire contents of which are incorporated by reference herein, and the above-identified publication by Pang et al. Referring to FIG. 2, a reference electrode 30 is shown held at a potential V_{ref} 34 with respect to the top electrode 16. As an example, the reference electrode 30 may be an oxidized silver wire (Ag/AgCl), and v_{ref} can be a few tens to about 100 mV positive at the electrode 16 side with respect to the reference 30 in the case that the top electrode 16 is made of palladium. A bias Vₜᵤₙ 36 may be applied across the device junction, with values of Vₜᵤₙ lying in the range of 50 mV to 800 mV (e.g., 300 mV). A transconductance amplifier 38 generates a signal proportional to the current flowing across the junction. This signal can be the electronic output of the device, but it may also serve to indicate the presence of a molecule in the reading junction, so it can be used to control the flow of the molecule past the reading junction. In some instances, the current signal from the transconductance amplifier 38 is sent to a computing device 42 where signal is both stored for interpretation in terms of molecular sequence, and as a means for detecting movement of the molecule past the reading head.

In some embodiments, a collector (i.e., control) electrode 18 on the underside of the device may be used. Since this electrode is part of the fabrication, a separate collector electrode for each sequencing device may be used even if many devices on a wafer are simultaneously in contact with a common electrolyte solution. If the polymer to be sequenced is negatively charged, then the collector electrode 18 may be biased positive relative to the tunneling electrodes 10, 16 so as to pull the polymer across the tunnel gap against the restraining force of the molecular ring 24. Since the stalling force of many molecular motors lies in the range of 10-30 pN (e.g., see “Single-Molecule Studies of RNA Polymerase: Motoring Along” by Herbert et al., Annual Review of Biochemistry, published Apr. 14, 2008 (online)) a polymer can be moved against the intrinsic friction of a molecular motor clamping it by a voltage of 40 mV to 125 mV. This is because, for a polymer of constant linear charge density, the force exerted by an electric field is about 0.24 pN/mv, as described by Keyser et al. (e.g., see “Direct force measurements on DNA in a solid-state nanopore” by Keyser et al., Nature Physics, published Apr. 14, 2008 (online)). The polymer may be pulled out of the molecular clamp in the first place by Vₜᵤₙ 36 which can significantly exceed the minimum voltages needed (of 40 to 125 mV). However, this may pull out the first residues of the polymer, because the vertical separation between the tunneling electrodes 16 and 10 is small (about 2 nm). The supporting substrate is preferably about 50 nm in thickness, so a positively charged electrode 18 on the underside of the device may pull out this much length of polymer, so long as the potential of the collector electrode 18 is more positive than that of the lower tunneling electrode 10. The field is generated by ion flow between electrodes 10 and 18. In the case of DNA, 50 nm corresponds to about 150 bases. This initial pulling on the polymer is achieved by transiently moving the potential of the electrode 18 to be 10 to 100 mV above the potential of electrode 10. For example, if Vₜᵤₙ is +300 mV, then V_{col} 40 would be set to +310 mV to +400 mV with respect to the common connection to the positive side of V_{ref} 34.

In some instances, in order to read even longer runs of sequence, a second reference electrode 32 is placed inside a lower reservoir of electrolyte, communicating with the upper reservoir only by means of the opening 20 in the device. After all the devices on a wafer have read the sequence of the polymer pulled through by the first application of a positive pulse to the individual collector electrodes (18 on each individual device) the polymers collected onto the collector electrodes 18 are pulled off by the application of a positive voltage V_{clr} to the second reference electrode 32. V_{clr} exceeds Vₜᵤₙ being in the range of 300 to 600 mV. The application of a significant potential difference between the reference electrodes 30 and 32 may also result in significant current flows from the tunneling electrodes 10 and 16. However, as the clearing pulse V_{clr} is applied only transiently between reads, it may not interfere with the readout of the sequence; sequence readout may occur on the next application of a positive bias to electrode 18. However, current between the electrodes 10, 16 and 18 can be small because of the passivation applied to the top and bottom of the device.

In some embodiments, the molecular clamp may be a helicase, an example of which is the T7 helicase. This helicase binds single stranded DNA spontaneously, translocating along the molecule at about 130 nt/s, hydrolyzing one deoxythymidinetriphophate (dTTP) per three nucleotides. When it encounters a double strand-single strand junction, its progress may be slowed to about 15 bp/s (e.g., as discussed in “Mechanochemistry of t7 DNA helicase” by Liao et al., J Mol Biol, published Jul. 15, 2005).

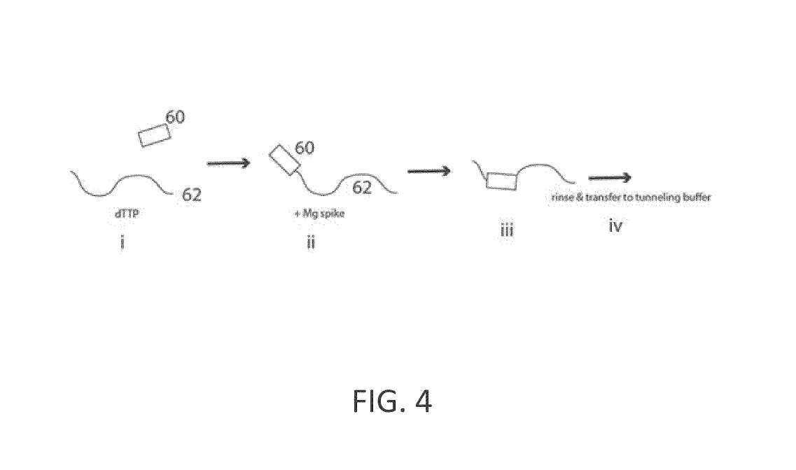

With reference to FIG. 4, an example embodiment of sample preparation for a DNA-helicase complex is shown. At step (i), a helicase 60 is first bound to single stranded DNA 62 in a helicase buffer (50 mM Tris-HCl (pH 7.6), 40 mM NaCl, and 10% (v/v) glycerol). In the presence of dTTP, the T7 helicase (e.g., T7 G4 pA from www.Biohelix.com) may bind the end of ssDNA, e.g., step (ii). At step (iii), the helicase can be advanced along the ssDNA using a pulse of Mg ions for a distance that can be controlled by the duration and concentration of the pulse, as described in the afore-mentioned publication by Liao. At step (iv), the DNA fragments, as bound by helicase, can then be filtered and re-suspended into a buffer suitable for tunneling measurements (e.g., 10 mM phosphate buffer, ph 7.0). The complex of helicase and ssDNA can be transferred into the upper electrolyte chamber of the device where the NHS ester can capture and react with an amine in the lysine residues exposed on the surface of the helicase. In some instances, a more specific site of capture may be readily achieved by inserting a cysteine into a recombinant helicase sequence and using, for example, vinylsulfone as the reactive group on the linker.

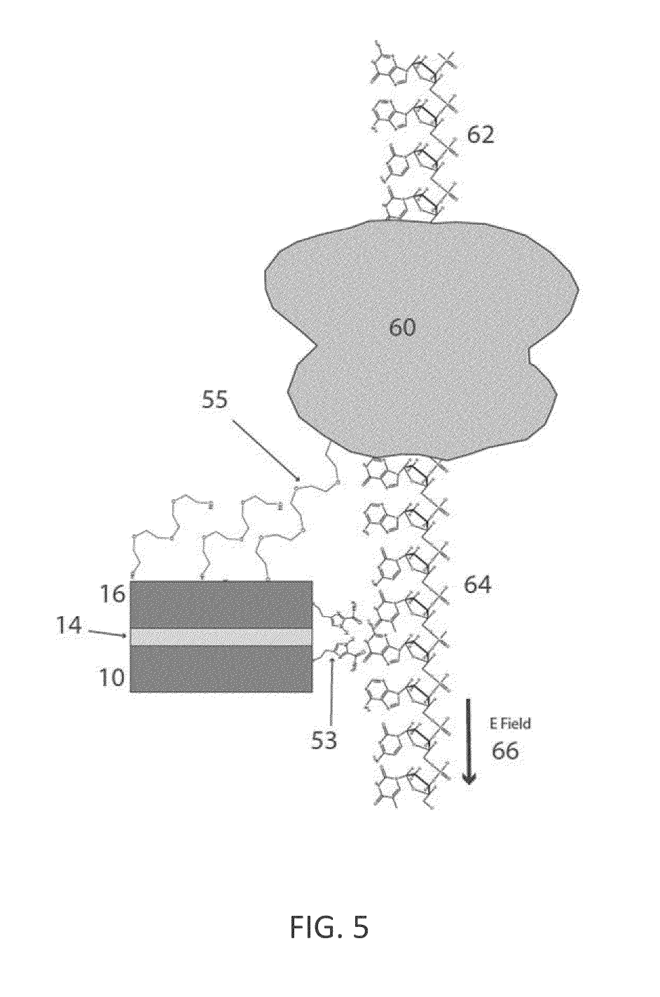

With reference to FIG. 5, a helicase 60 may become attached to the top electrode 16 in an exposed region (e.g., a few tens of nm in extent) at the top of electrode, which may be left exposed as a result of over etching of the masking material by the several cycles of RIE required to make the device. In some embodiments using single stranded DNA, either end of the captured DNA (62 or 64) could be drawn further into the device by the electric field 66 applied by the collector electrode 18.

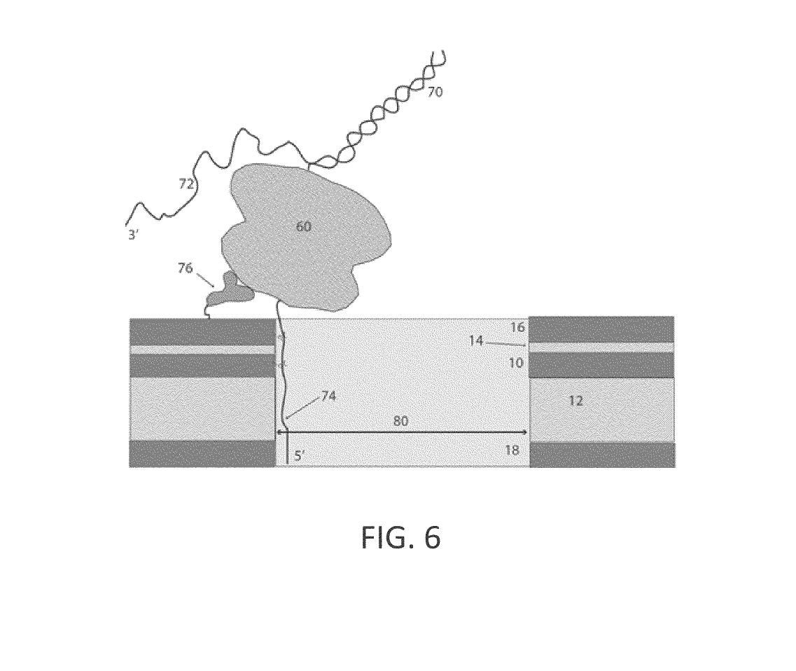

With reference to FIG. 6, in some embodiments, a nanopore of finite diameter is shown, serving to restrict the entry of the double stranded region into the reading section of the device. In this embodiment, the use of an antibody as a reversible tether for the helicase is also illustrated. Double stranded DNA 70 may be complexed with helicase 60, following same or substantially similar procedure outlined for single stranded DNA with reference to FIG. 4. In some instances, the duration of the Mg pulse may determine the amount of unwinding of the DNA into 3′ (72) and 5′ (74) ended strands, with the orientation of the helicase determining which strand is more likely to enter the device. One way to control this is to use a site-specific modification as described above. Another way may be to use an antibody 76 raised to a specific epitope on the surface of the helicase (the helicase is a heptamer, but equivalent sites at the bottom end of the molecule can be used, for example). In some instances, the strand to be read may be pulled into the junction using the electric field between the bottom tunneling electrode 10 and the collector electrode 18 to unwind the DNA by force alone. This may be useful in avoiding the use of dTTP for active transport, as this nucleotide (and its reaction product dTDP) may bind the recognition molecules in the tunnel junction, creating a spurious background.

In some instances, referring to FIG. 6, the presence of the dangling 3′ strand 72 and the double stranded region 70 may bring about some complications. For example, as and when the dangling 3′ strand becomes long enough to enter the device, it may alter the speed of unwinding of the DNA as more forces are applied to across the helicase because both the 3′ and 5′ strands are pulled on. In addition, the signal from the device may reflect the presence of two molecules. However, this may be readily identifiable and decodable by a computer algorithm, particularly because the two sets of reads may be complementary and shifted in linear distance along the DNA by an amount similar to the diameter of the helicase molecule. In some instances, translocation could be stopped altogether if the double stranded region is pulled into the opening. This can be prevented by keeping the largest dimension of the opening 80 to less than the persistence length of double stranded DNA (which is 50 nm). Since RIE can be used to make openings as small as 20 nm in diameter, this condition can be readily achieved.

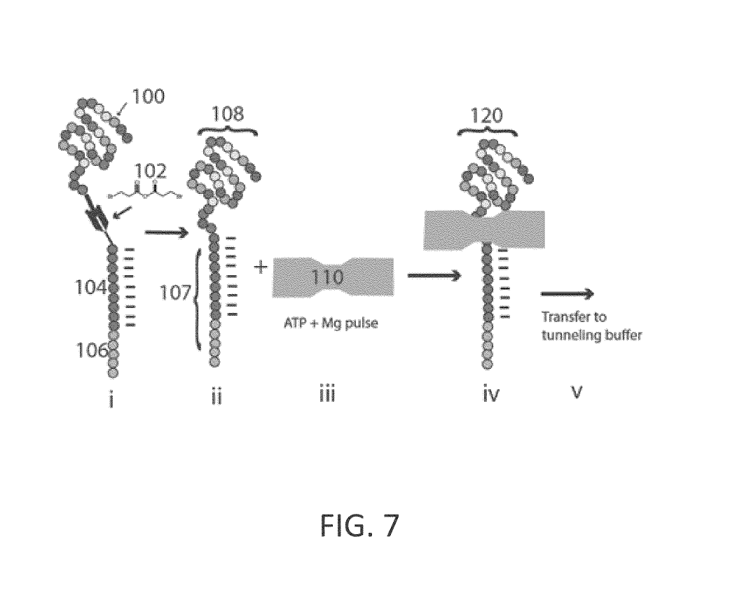

With reference to FIG. 7, an example embodiment of sample preparation for a protein sequencing is shown. In some instances, the device as disclosed herein may read protein sequence. Proteins can be readily complexed with a molecular motor (e.g., the ClpX motor from e-Coli) by ligating a recognition sequence onto the end of the protein (e.g., the so-called ssrA tag in the case of ClpX). A method for attaching a tail comprised of charged amino acids and the ssrA tag to proteins is described in the aforementioned WIPO Patent Application No. WO 2014/190299. An example embodiment of sample preparation for protein sequencing may use bromoactyl anhydride 102 as a coupling agent to conjugate the protein 100 to be sequenced to an amino acid sequence rich in negatively charged residues 104, e.g., step (i). The tail may be terminated in the ssrA sequence for recognition by ClpX. In some instances, other sequences may be be used for other unfoldase molecules. Step (ii) shows a conjugated protein 108 and tail 107 comprising both the charged region and the recognition sequence. At step (iii), the complex 108 may be incubated with ClpX protein and ATP and subjected to a pulse of Mg ions in order to advance the ClpX motor to the junction with the protein to be sequenced. At step (iv), the resulting complex may be captured by filtration, rinsed and transferred to tunneling buffer.

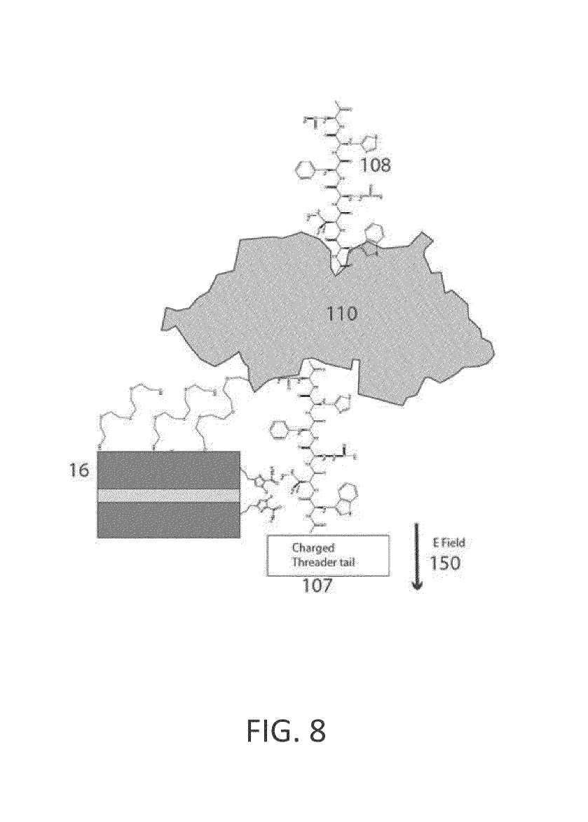

With reference to FIG. 8, an example embodiment of molecular components for reading protein sequence using an unfoldase motor and a protein modified at its N terminus with a series of charged residues and a binding recognition sequence for the unfoldase is shown. In some implementations, using one of the coupling reactions described above for helicase motors, the ClpX 110—protein 108 complex may be tethered to the top electrode 16. In some instances, a bias applied to the collector electrode 18 generates an electric field 150 that exerts a force to pull the protein into the device by means of a force applied to the tail-ssrA peptide 107. In the case of a protein carrying few intrinsic charges, the distance between the bottom reading electrode 10 and the collector electrode 18 may determine the length of chain that can controllably be moved through the gap. As such, it may be advantageous to use a thicker membrane 12 for protein sequencing.

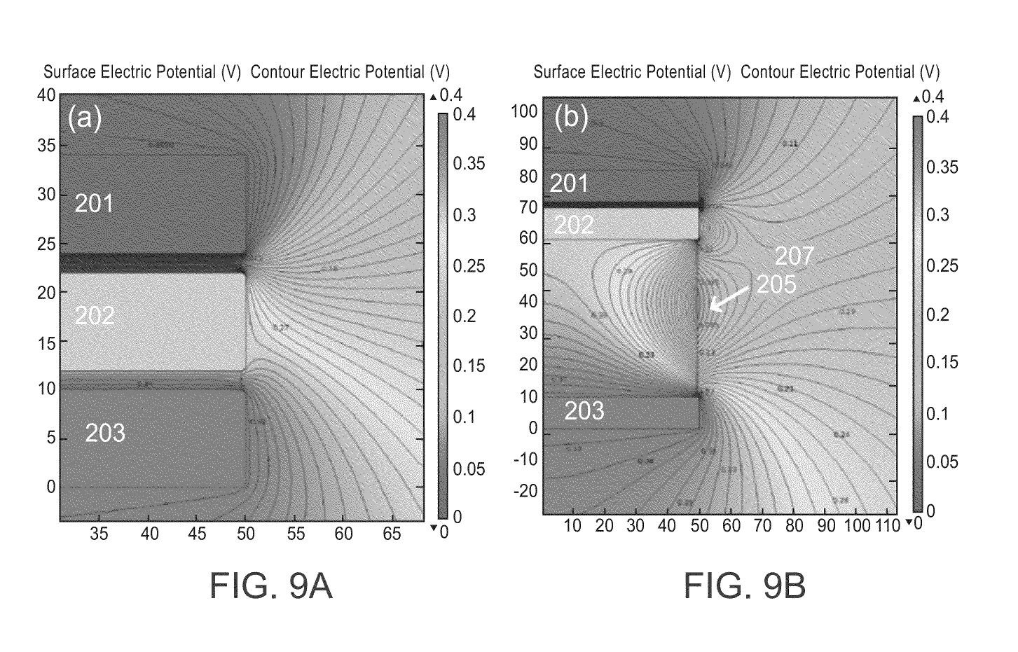

With reference to FIGS. 9A-B, in some embodiments, example potential distributions near tunneling electrodes due to the placement of a collector electrode are shown. In some instances, the distance of the collector electrode 203 from the top tunneling electrode 201 and/or the bottom tunneling electrode 202 may influence the probability of capture of a charged polymer by the collector electrode 203. For example, although it may be desirable to have the collector electrode 203 a longer distance from the tunneling electrodes 201 and 202 to pull out a longer amount of charged polymer (or pull a charged tail attached to neutral polymer further), the electric field in the region between the tunneling electrodes 201 and 202 and the collector electrode 203 may become screened as the tunneling electrodes 201 and 202 and the collector electrode 203 become separated by a distance much larger than the Debye length (e.g., about 10 nm in about 1 mM salt solution such as NaCl). In some instances, distance may be measured from some convenient reference points on/close to the electrodes. FIGS. 9A and 9B show the electric potential inside and outside the device when the distance between the collector electrode 203 and the tunneling electrodes is about 2 nm and 50 nm, respectively (in these examples, the top tunneling electrode 201 is held at 0V, the bottom tunneling electrode 202 at +300 mV and the collector electrode 203 at +400 mV). In these examples, when the collector electrode 203 is close to the bottom tunneling electrode 202 (e.g., FIG. 9A), the potential becomes increasingly positive in passing from the top of the device to the bottom of the device, while when the distance between the collector electrode 203 and bottom tunneling electrode 202 is increased to 50 nm (e.g., FIG. 9B) the potential becomes less positive in passing from the bottom tunneling electrode 202 towards the middle of the dielectric layer on the face of the device, e.g., 205. In some instances, this may present a barrier to the motion of a negatively charged polymer. In some instances, there may exist a region 207 of relatively constant potential a little further from the surface, which may facilitate the diffusion of the polymer into the region of higher positive potential. However, the probability of capture by the bottom electrode may decrease as the separation between tunneling electrodes 201 and 202 and collector electrode 203 is increased further.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Moreover, some embodiments are distinguishable from the prior art by lack of or elimination of structure, functionality and/or a step specifically disclosed in the prior art (e.g., some embodiments may be claimed with negative limitations to distinguish them from the prior art).

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms. In this regard, references to publications in the detailed description are included to provide, at least for some embodiments, a supporting and enabling disclosure, as well providing additional disclosure that when combined with one and/or another disclosed inventive subject matter provide yet additional embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A device for reading polymer sequence, comprising:
a first electrode;
a molecular clamp for holding polymer to be sequenced to the first electrode;
a second electrode separated from the first electrode by a first dielectric layer; and
a third electrode, separated from the second electrode by a second dielectric layer, configured to be biased with respect to the first electrode and/or the second electrode so as to cause the polymer to flow across a junction between the first electrode and second electrode against a restraining force of the molecular clamp.

2. The device of claim 1, wherein the molecular clamp is selected from the group comprising a helicase, a polymerase, a polymerase clamp protein, a topoisomerase and a cyclodextrin that have a DNA molecule threaded through them during the process of reading the DNA sequence.

3. The device of claim 1, wherein the molecular clamp is an unfoldase protein.

4. The device of claim 1, wherein the first dielectric layer forms a gap ranging in thickness from about 0.5 nm to about 5 nm between the first electrode and the second electrode.

5. The device of claim 1, wherein the second dielectric layer forms a gap exceeding 1 nm in thickness between the second electrode and the third electrode.

6. The device of claim 1, wherein current flowing between a pair of electrodes selected from the group comprising the first electrode, the second electrode and the third electrode is read by applying bias between the pair of electrodes.

7. The device of claim 1, further comprising recognition molecules, tethered to one or more electrodes, configured to form complexes with molecular regions of the polymer to be sequenced.

8. The device of claim 1, wherein voltage between a pair of electrodes selected from the group comprising the first electrode, the second electrode and the third electrode controls the flow of the polymer across the junction.

9. The device of claim 1, wherein one of the first electrode, the second electrode, and the third electrode is configured to be held at a constant bias with respect to a reference electrode in contact with a solution containing the polymer to be sequenced.

10. The device of claim 1, further comprising a second reference electrode configured to be charged to a voltage with respect to one of the first electrode, the second electrode, and the third electrode so as to advance the polymer across the junction.

11. The device of claim 1, further comprising a passivating layer covering a substantial portion of a surface of one of the first electrode, the second electrode, and the third electrode.

12. The device of claim 1, wherein the first electrode, the second electrode, and the third electrode are selected from the group comprising palladium, platinum, and gold.

13. The device of claim 1, wherein the first dielectric layer and the second dielectric layer are selected from the group comprising silicon nitride, any one of oxides of silicon, any one of oxides of hafnium, and aluminum oxide.

14. A method for reading polymer sequence, comprising:
providing a system comprising a first electrode, a molecular clamp for holding polymer to be sequenced to the first electrode, a second electrode separated from the first electrode by a first dielectric layer; and a third electrode, separated from the second electrode by a second dielectric layer; and
biasing the third electrode with respect to the first electrode and/or the second electrode so as to cause the polymer to flow across a junction between the first electrode and second electrode against a restraining force of the molecular clamp.
